(19) United States
(12) Patent Application Publication
(10) Pub. No.: US 2016/0139110 A1
(43) Pub. Date: May 19, 2016

(54) FLUID CHANNEL SYSTEM FOR EXAMINING CELLS

(71) Applicant: ibidi GmbH, Martinsried (DE)

(72) Inventors: Roman Zantl, Baldham (DE); Miriam Dietrich, Munchen (DE)

(21) Appl. No.: 14/802,193

(22) Filed: Jul. 17, 2015

(30) Foreign Application Priority Data
Nov. 14, 2014 (EP) 14193245.9

(51) Int. Cl. G01N 33/50; G03F 7/30

(52) U.S. Cl. CPC G01N 33/5029; G03F 7/30

(57) ABSTRACT

A fluid channel system for examining cells may comprise a chamber filled with at least one photopolymerized hydrogel and/or one polymerized photodepolymerizable hydrogel to at least 90% capacity. The chamber may open exclusively towards at least one fluid channel via at least two openings. The at least one fluid channel and the chamber may each be in the form of a hollow space in a substrate, and each fluid channel may have two openings to the outside. In an embodiment, the system may comprise a hollow space in a substrate with at least one photopolymerized hydrogel and/or one polymerized photodepolymerizable hydrogel may be arranged and structured such that at least one fluid channel and a coherent hydrogel structure are formed. The hydrogel structure may adjoin the at least one fluid channel at two separate surface areas and connect to the outside exclusively via the at least one fluid channel.

### TECHNICAL FIELD

The invention relates to a fluid channel system for examining cells.

### BACKGROUND

The behavior of cells depends on ambient conditions, such as the concentration of nutrients, salts, the pH value, temperature, growth factors and the concentrations of certain gases, e. g. oxygen. Further important parameters are the dimensionality in which the cells are located, the communication with other cells which have to be arranged in the direct vicinity in a defined manner for this purpose, and the stiffness of the surfaces or matrices with which the cells interact, e. g. via adhesion. The creation of conditions that are similar to those in the living organism is a current challenge in cell biology. This in particular serves to examine the strength of drugs or also basic scientific questions under ambient conditions that are as relevant as possible and similar to in-vivo conditions. Hydrogels are in many respects well suited to simulate mechanical and hydrodynamic conditions in tissue.

It is known from prior art to cultivate cells in three-dimensional hydrogels arranged in fluid channels whose stiffness is adaptable to that of the cell-specific ambient parameters in the organism, among other things for examining cells which are immobilized in hydrogels.

For example, Heo et al., A Microfluidic Bioreactor Based on Hydrogel-Entrapped E. coli: Cell Viability, Lysis, and Intracellular Enzyme Reactions” (Anal. Chem. 2003, 75, 22-26) describes how E. coli cells are enclosed in hydrogel micropatches that are photopolymerized in microfluidic systems. These micropatches have dimensions of 100 μm to 500 μm. Small molecules in a solution surrounding the hydrogel diffuse into the gel and hit the cell. Similar systems are also known for detecting antibodies. For example, Ikami et al. (Immuno-pillar chip: a new platform for rapid and easy-to-use immunoassay, Lab Chip, 2010, 10, 3335-3340) shows hydrogel columns that are formed in a microchannel by photopolymerization and in which anti-CRP antibody molecules are immobilized.

The advantage of the above-described systems is that the channels themselves may be very simple. However, the problem is that just in the examination of cell growth, the shown hydrogel columns or hydrogel micropatches are too small structures to realistically imitate the conditions in real tissue.

Alternative channel systems, in which even larger hydrogel sections are possible, are described, for example, in Shin et al. “Microfluidic assay for simultaneous culture of multiple cell types on surfaces or within hydrogels” (Nature Protocols, Vol. 7 No. 7, 2012, 1247-1259). This publication describes how three-dimensional cell cultures are examined by introducing them into hydrogel located in chambers between two microchannels. A similar structure is also described in Chung et al. “Cell migration into scaffolds under co-culture conditions in a microfluidic platform” (Lab Chip, 2009, 9, 269-275). In both cases, the setup is relatively complicated because the liquid hydrogel, which is introduced into the chamber through feed openings especially provided for this purpose, must not flow into the channels. Therefore, the chamber must be designed such that the liquid hydrogel is retained in the chamber just by surface tension. In both cases, columns are used for this purpose which are arranged in the region where the hydrogel is to be fixed.

As already mentioned, larger hydrogel sections are also possible here, however, the system is relatively complicated since feed openings and elements for increasing surface tension must be provided for each chamber.

It is therefore an object of the invention to provide a fluid channel system for examining cells which permits to provide as realistic boundary conditions as possible for cell growth while being of a simplified structure. This object is achieved by the subject matter of the independent patent claims.

### BRIEF SUMMARY

The inventive fluid channel system for examining cells comprises a chamber which is filled with at least one photopolymerized hydrogel and/or one polymerized, photodepolymerizable hydrogel to at least 90% of capacity, in particular at least 95%, in particular completely, the chamber being open exclusively towards at least one fluid channel via at least two openings, wherein the at least one fluid channel and the chamber are each embodied in the form of a hollow space in a substrate and wherein each fluid channel has two openings to the outside.

A further inventive fluid channel system for examining cells comprises a hollow space in a substrate, wherein at least one photopolymerized hydrogel and/or one polymerized photodepolymerizable hydrogel is arranged in the hollow space, the hydrogel being structured in each case such that at least one fluid channel and a coherent hydrogel structure are formed, and wherein the hydrogel structure adjoins the at least one fluid channel at two separate surface areas and has a connection to the outside exclusively via the at least one fluid channel.

The above mentioned inventive fluid channel systems each have the advantage that their structure is comparatively simple and nevertheless realistic boundary conditions for cell growth are provided.

In particular, when using the above-mentioned fluid channel system, the supply of cells in a tissue in the body may be imitated because a liquid with particles may be filled into the at least one fluid channel and a pressure may be applied across the openings to the outside which is similar to the interstitial pressure in the body and promotes the penetration of the particles into the hydrogel.

Each fluid channel has two openings to the outside and at least one opening to the chamber. In the term “openings to the outside”, the word “outside” relates to the region outside the substrate. The at least two openings of the chamber or the two separate surface areas are embodied non-coherently. The openings of the chamber may be separated, for example, by wall pieces which may be embodied, for example, in the substrate.

If applicable and not otherwise specified, features which are described below only for the openings of the chamber are also applicable to the surface areas.

The fact that the chamber is exclusively opened towards the at least one fluid channel means that it does not have a direct connection to the outside but has a connection to the outside exclusively via the at least one fluid channel.

The chamber may be open towards a first fluid channel via exactly one opening and towards a second fluid channel via exactly one opening, or it may be open towards the exactly one fluid channel via exactly two openings. The chamber may be delimited from the at least one fluid channel by an interrupted wall which may be formed, for example, in the substrate. Openings in the chamber towards the at least one fluid channel then designate the holes or interruptions in the interrupted wall. Otherwise, the chamber is considered to be limited by the continuation of the fluid channel wall or fluid channel walls which may also be embodied in the substrate. Openings of the chamber to the at least one fluid channel then designate the interruption in the fluid channel wall or the fluid channel walls.

The at least one fluid channel may comprise exactly one fluid channel, where the chamber is open towards the fluid channel at a first partial area of the fluid channel and at a second partial area of the fluid channel. The at least one fluid channel may comprise exactly one fluid channel, where one of the two surface areas of the hydrogel structure adjoins a first partial area of the fluid channel and the other one of the two surface areas of the hydrogel structure adjoins a second partial area of the fluid channel.

This means, one opening each of the chamber or one surface area may be arranged at each partial area of the fluid channel. In particular, two of the at least two openings of the chamber or the two surface areas may be opposite.

If the fluid channel system comprises exactly one fluid channel, the latter must be bent or angled in its extension between two openings of the chamber or between two surface areas of the hydrogel structure such that the chamber or the hydrogel structure is at least partially arranged between the two partial areas. For example, the fluid channel may be U-shaped or V-shaped. The partial areas may be embodied, for example, in the form of legs.

The at least one fluid channel may comprise a first fluid channel and a second fluid channel, where the at least two openings of the chamber comprise a first opening to the first fluid channel and a second opening to the second fluid channel. The at least one fluid channel may comprise a first fluid channel and a second fluid channel, wherein the at least two separate surface areas comprise a first surface area adjoining the first fluid channel and a second surface area adjoining the second fluid channel. If the at least one fluid channel comprises a first fluid channel and a second fluid channel, the chamber or the hydrogel structure may be completely or partially arranged between the first and the second fluid channels.

The at least one hydrogel may have the same shape as or a shape different from the chamber. The chamber may be filled with hydrogel over its total height. In addition or as an alternative, the chamber may be filled with hydrogel over its complete length. As an alternative or in addition, the chamber may be filled with hydrogel over its complete width. The hydrogel may in particular extend to all walls of the chamber embodied in the substrate. As an alternative, the hydrogel may not extend to all walls of the chamber embodied in the substrate.

At least 80%, in particular at least 90%, in particular at least 95%, in particular 100% of the hydrogel in the chamber may be coherent.

The hydrogel in the chamber or the hydrogel structure may be structured, in particular structured such that channels are formed in the hydrogel or in the hydrogel structure, in particular channels which connect the first fluid channel and the second fluid channel or the one partial area of the single fluid channel with the other partial area of the single fluid channel to each other. Such channels may also be branched. Thus, the supply of particles from a solution from the fluid channel or from one of the fluid channels into the hydrogel may be increased. Furthermore, cells may optionally grow along the channels. As an alternative or in addition, hollow spaces which may also be interconnected may be present in the hydrogel in the chamber or in the hydrogel structure. It may be advantageous for some experimental setups for the liquid migrating through the hydrogel to accumulate in hollow spaces.

The at least one hydrogel or the hydrogel structure may comprise at least two different hydrogels. The hydrogels may in particular have different properties with respect to the diffusion of molecules, cell growth and liquid permeability.

The hydrogel, or at least one of the hydrogels, in particular all hydrogels, or the hydrogel structure, may comprise cells and/or cell accumulations, for example cell spheroids. The cells may be, for example, tumor cells and/or endothelial cells and/or kidney cells and/or liver cells and/or neurons.

The at least one hydrogel or the hydrogel structure may be transparent or translucent in the polymerized condition. The at least one hydrogel or the hydrogel structure may be migratable or non-migratable for cells. Those hydrogels are referred to as migratable in which cells, in particular tumor cells, fibroblasts, endothelial cells, migrate within a period of 10 hours by one cell diameter (for example a mean cell diameter for this cell type) into any direction (also referred to as migration speed).

The at least one hydrogel or the hydrogel structure may comprise a synthetic hydrogel. The at least one hydrogel or the hydrogel structure may comprise a hydrogel which is polymerized by covalent cross-linking Examples of this are hydrogels comprising polymethyl methacrylate or, in particular functionalized, polyethylene glycol (PEG). PEG is inexpensive and admitted for the use in drugs. PEG is moreover classified as being very biocompatible. The PEG monomers may be provided at their ends with functional groups, such as acrylic acid esters, methacrylic acid esters, vinyl carbonates, vinyl carbamates or other reactive En-functionalities. These react with each other under the influence of light and the use of a photoinitiator via a radical chain reaction and form cross-linked hydrogels.

Furthermore, the at least one hydrogel or the hydrogel structure may comprise copolymers, for example with thiol groups as reactive species. For example, multi-arm PEG monomers react with norbornene end groups as reactive En-component with the thiol groups into a thio-ether via a radical click reaction. Thus, a particularly homogenous mesh size may be obtained. Moreover, by varying the molecular weight of the employed monomers, the amount of monomers used, and the ratio of different monomers with respect to each other, the elasticity, swelling behavior and mesh size of the hydrogel may be controlled. The elasticity (G′) of the hydrogel, expressed in form of the Young's modulus, may be, for example, between 0.1 to 100 kPa, in particular between 0.05 and 40 kPa, in particular between 0.05 and 10 kPa, in particular between 0.1 and 3 kPa. The swelling behavior may be quantized by the swelling ratio, i. e. the ratio of the mass of the swollen hydrogel to the mass of the dried gel, where the swelling ratio may be between 5 and 100, in particular between 5 and 50, in particular between 10 and 40. The mesh size may be between 10 nm and 60 nm. By the use of monomer mixtures, moreover various functions may be incorporated into the hydrogel. The mesh size is used corresponding to common definition, i. e. as the distance between two adjacent knots, measured from the knot center to the knot center, where the cross-linking points in the hydrogel in this case correspond to the knots.

The photodepolymerizable hydrogel may comprise a spontaneously gelling gel. Spontaneous polymerization may be achieved, for example, by means of temperature reduction (for example with agarose) or temperature increase (for example with matrigel) or pH modification (for example with collagen 1).

A hydrogel may be simultaneously photopolymerizable and photodepolymerizable, depending on the wavelength. Polymerization and depolymerization then each are effected at exposures with light of different wavelengths.

For the polymerization of the hydrogels, acrylates may be used which interact via redox activation. Click reactions with azides and cyclooctine derivatives are also possible. If simple alkines are used instead of cyclooctines, an additional activation with copper catalysts may be effected. An oxidation of thiols may also be used for polymerization by disulfide bridges being formed.

For the depolymerization of hydrogels by light, nitrobenzoyl groups may be used which, for example, absorb light within a range of 325 to 415 nm and are split in the process. Hydrogels that are formed by disulfide bridges may also be photodepolymerized under the influence of a photoinitiator. Coumarin derivatives form a further photodepolymerizable group.

Via a thiol-en click reaction with a photoinitiator and an exposure at 450 to 550 nm, a molecule may be bound to the hydrogel which contains a nitrobenzoyl group. Thereby, both the connection (450 to 550 nm) and the detachment (365 nm) of the molecule by exposure at the corresponding wavelength are possible.

Peptide sequences containing one or several cysteines or other polythiol molecules may be covalently integrated into the hydrogel by the click reaction. For example, by the incorporation of different RGD-containing peptide sequences, cell adhesion to hydrogels may be examined. Further functions of the extracellular matrix, such as the degradability, for example by matrix metalloproteases (MMP), may be imitated by the use of suited peptide sequences. In the hydrogel, hydrolysis-sensitive groups, such as polylactate (PLA) or thio-ether, which are formed, for example, by the reaction of PEG diacrylate (PEGDA) with dithiothreitol (DTT), may be contained at the beta position to an ester function. These may lead to a continuous degradation of the hydrogel.

The substrate may be plate-like. Plate-like in particular means that the height of the substrate is clearly smaller than its length or its width, the ratio of height to width or to length being in particular smaller than 0.5, preferably smaller than 0.3, preferably smaller than 0.15.

It should be noted that in this application, when ratios are stated, the first mentioned quantity represents the numerator and the second quantity the denominator.

The substrate may be of a single-piece or multi-piece design. The substrate may in particular comprise at least one base plate and one cover plate. In its intended use, the base plate is arranged at the bottom, i. e. underneath the cover plate. The base plate, based on its largest area, may be smaller than the cover plate, based on its largest area. The cover plate and/or the base plate may comprise side walls. As an alternative or in addition, side walls may be present additionally to the base plate and the cover plate. The side walls may be perpendicular to the base plate and/or the cover plate.

The openings to the outside may be embodied in the form of through holes in the substrate. The openings to the outside may be embodied in any side of the substrate, in particular all in exactly one side of the substrate. The openings to the outside may be designed such that they face upwards when used as intended. The openings to the outside may be present in the cover plate and/or the base plate of the substrate, in particular exactly all in the cover plate. If side walls are not part of the cover plate and the base plate, the openings to the outside may be embodied, as an alternative or in addition, in the side walls.

The cover plate may have a single-piece design, and a first side (one of the two largest areas) of the cover plate may comprise the top and the side walls of the at least one fluid channel or the side walls of the hollow space. Additionally, the cover plate may comprise the openings to the outside in the form of through holes. The cover plate may comprise top parts or reservoirs which surround the openings to the outside. These top parts or reservoirs may in particular be formed on the second side opposite the first side. The top parts or reservoirs are connected to the at least one fluid channel via the openings to the outside. This permits to introduce liquids into the at least one fluid channel in a suited manner. On such a cover plate, a base plate, in particular in the form of a plastic foil, may be fixed.

The top parts or reservoirs may in particular be designed such that they may be connected to hoses or tubes. The top parts or reservoirs may in particular be embodied as connections for a pump, for example an air pump. The top parts or reservoirs may correspond, for example, to a female Luer adapter at the inner side.

At least one part of the substrate, in particular the base plate, may have a thickness between 1 μm and 2 μm, in particular between 50 μm and 300 μm, in particular between 100 μm and 250 μm, in particular 140 μm to 170 μm. For example, borosilicate glass may be used as base plate, e. g. with a thickness between 120 μm and 190 μm, in particular with 140 μm or 170 μm. The cover plate may have a thickness of greater than or equal to 1 mm.

The substrate or one or all parts of the substrate may be embodied in the form of molded parts, or be embodied by applying and/or removing material and/or connecting several components. In particular, indentations may be formed in one plastic piece which are covered with a plastic foil. For example, the channel and the chamber may be formed in this manner.

The substrate may comprise a glass plate, for example of borosilicate glass, and/or a plastic plate and/or a plastic foil. Possible plastics are, for example, COC (cyclo-olefin copolymer), COP (cyclo-olefin polymer), PE (polyethylene), PS (polystyrene), PC (polycarbonate) or PMMA (polymethyl metacrylate). In particular, the base plate may be embodied in the form of a plastic foil.

At least a part of the substrate, in particular the base plate and/or the cover plate of the substrate, may be transparent, in particular in a region above and underneath the chamber or the hydrogel structure. The substrate, in particular the base plate and/or the cover plate of the substrate, may comprise plastic, in particular without double refraction and/or with an autofluorescence that is essentially equal to the autofluorescence of a conventional cover glass, in particular in a region above and underneath the chamber or the hydrogel structure.

The above-mentioned properties are particularly advantageous for optical applications, in particular for precise photopolymerization and for high-resolution microscopy.

If the substrate is a multi-piece substrate, the components may be connected to each other by means of adhesive agents, solvents, UV-treatment, radioactive treatment, laser treatment or thermal bonding. Thermal bonding may have been obtained all over the surface or in strips, in particular only along the edge of the substrate. This advantageously permits a firm connection.

The substrate may be completely or partially functionalized to bind the polymerized hydrogel or the hydrogel structure in particular chemically to the substrate. Functionalization increases the adherence of the hydrogel to the substrate.

The sum of the surfaces of all openings of the chamber or the two surface areas towards the at least one fluid channel may be between 0.002 mm² and 200 mm², in particular between 0.05 mm² and 5 mm², in particular between 0.2 mm² and 1.6 mm². In particular, the surface of all openings of the chamber or the surface area towards one partial area each of the exactly one fluid channel, or the surface of all openings of the chamber or the surface area towards one of the two fluid channels can be each between 0.001 mm² and 100 mm², in particular between 0.1 mm² and 10 mm², in particular between 0.4 mm² and 3.2 mm². The size of each individual opening may be between 0.0005 mm² and 50 mm², in particular between 0.025 mm² and 8 mm², in particular between 0.2 mm² and 1.6 mm².

The above mentioned features permit in each case to structurally influence the amount of particles which penetrate from a liquid in the at least one fluid channel into the hydrogel. A large surface in particular permits a high penetration rate of particles into the hydrogel.

The volume of the chamber or the hydrogel structure may be embodied, for example, as a cuboid, a cube, a prism, a cylinder, a cone, a pyramid, a truncated cone or a truncated pyramid. If the volume is embodied as a prism, it may in particular be a prism with triangular, quadrangular or any other polygonal base. The base is the area of the bottom of the chamber or the interface between the hydrogel structure and the substrate.

In particular, the chamber may have a top and a bottom which are each formed by the parallel cover plate and base plate of the substrate. Their side walls may be perpendicular with respect to the bottom or inclined at an angle of up to +/−20°, in particular up to +/−10°, in particular up to +/−5° with respect to the bottom.

With a chamber or a hydrogel structure, their dimensions may be described by their width, length and height. The height of the chamber may be between 0.01 mm and 1 mm, in particular between 0.1 mm and 0.5 mm, in particular between 0.1 mm and 0.4 mm. The length of the chamber may be between 0.1 mm and 50 mm, in particular between 0.5 mm and 10 mm, in particular between 1 mm and 4 mm. The width of the chamber may be between 0.05 mm and 20 mm, in particular between 0.2 mm and 5 mm, in particular between 0.5 mm and 1 mm.

The ratio of the height to the width of the chamber or the hydrogel structure may be between 0.0005 and 20, in particular between 0.02 and 2.5, in particular 0.2. The ratio of the height of the chamber or the hydrogel structure to the length of the chamber or the hydrogel structure may be between 0.0002 and 10, in particular between 0.01 and 1, in particular 0.2. The height, length and width may be spatially constant or variable. If they are spatially constant, there is the special case of a cuboid-like chamber or hydrogel structure.

The width and length are extensions in a plane parallel to the base of the substrate, in particular the base plate, where the length corresponds to the extension in a direction along the fluid channels and the width corresponds to the extension in a direction perpendicular to the fluid channels. The height is measured in a plane perpendicular to the base of the substrate. The base of the substrate is the surface which faces downwards when used as intended.

The volume of the chamber or the hydrogel structure may be between 0.00005 mm³ and 1000 mm³, in particular between 0.01 mm³ and 25 mm³, in particular between 0.1 mm³ and 1.6 mm³.

The above-described height ranges permit several cell layers to be disposed one upon the other, so that a three-dimensional cell tissue is formed which still has a sufficiently small thickness, such that the cell sample may be microscopized similar to a two-dimensional cell sample. So, there is the advantage of being capable of observing three-dimensionally arranged cells which may behave physiologically naturally in a sufficiently thin layer.

The at least one fluid channel may have a length between 0.5 mm and 70 mm, in particular between 5 mm and 60 mm, in particular between 15 mm and 50 mm. The at least one fluid channel may have a width between 0.05 mm and 10 mm, in particular between 0.5 mm and 8 mm, in particular between 1 mm and 5 mm. The at least one fluid channel may have a height between 0.01 mm and 2 mm, in particular between 0.1 and 1 mm, in particular between 0.2 mm and 0.5 mm.

The ratio of the width of the at least one fluid channel each to the length of the at least one fluid channel may be between 0.0007 and 20, in particular between 0.008 and 1.6, in particular between 0.02 and 0.3, in particular 0.2.

The definition of top, bottom, length, width, and height of the at least one fluid channel corresponds to the definition of top, bottom, length, width, and height of the chamber or the hydrogel structure.

The at least one fluid channel may have each a round or oval cross-section or a polygonal cross-section (in a plane perpendicular to the base of the substrate), in particular a rectangular or trapezoidal cross-section. The at least one fluid channel may be non-branched or comprise one or several branches. The shape of the cross-sectional area may be constant or variable along the at least one fluid channel. Various cross-sections may be advantageous depending on the manufacturing process and the intended examination process.

In a plan view or a bottom view, each fluid channel may each have a polygonal, in particular rectangular or trapezoidal contour. The at least one fluid channel may have a straight, bent or angled extension. If the fluid channel system comprises two fluid channels, these may extend in parallel, in particular in the region of the chamber or the hydrogel structure.

For example, the at least one fluid channel may be limited by side walls, a bottom and a top. The inside volume may be, for example, cuboid-shaped. The side walls may be perpendicular with respect to the bottom of the at least one fluid channel, or inclined at an angle of up to +/−20°, in particular up to +/−10°, in particular up to +/−5° with respect to the bottom of the respective fluid channel. This may facilitate molding in the manufacture of the fluid channel by means of injection molding. As an alternative, each fluid channel may be embodied in the form of a round or oval tube.

The cross-section of the at least one fluid channel may each have an area between 0.005 mm² and 20 mm², in particular between 0.05 mm² and 8 mm², in particular between 0.2 mm² and 2.5 mm². Thus, the size of the at least one fluid channel may be adapted, for example, to the viscosity of the liquid hydrogel or the solutions that are used later.

Each fluid channel may be free from hydrogel.

The chamber or the hydrogel structure may be completely arranged between a first and a second fluid channel or be completely surrounded by the single fluid channel in three directions.

The ratio of the length of the chamber or the hydrogel structure to the length of the at least one fluid channel may be between 0.001 and 0.75, in particular between 0.1 and 0.7, in particular between 0.2 and 0.6, in particular 0.5. This permits to bring the chamber or the hydrogel structure into contact with solution over its complete length.

The height of the at least one fluid channel may be larger or equal to the height of the chamber or the hydrogel structure. The ratio of the height of the chamber or the hydrogel structure to the height of the at least one fluid channel may be between 0.01 and 1.5, in particular between 0.1 and 1, in particular between 0.2 and 0.5.

The above dimensions permit to bring the chamber or the hydrogel structure into contact with solution over its complete height.

In the fluid channel system, the two openings to the outside of the at least one fluid channel may be arranged along the at least one fluid channel so as to be opposed to each other, in particular at opposed ends of the at least one fluid channel.

Such an arrangement of the openings to the outside permits to introduce liquids, for example non-polymerized hydrogels or solutions, into the complete fluid channel and flush them out again.

In the single fluid channel, in the region between the two partial areas of the fluid channel, a fluidic resistance may be arranged. Thus, a pressure difference between the two openings of the chamber or between the two surface areas may be generated if a liquid is introduced under pressure into one of the openings to the outside.

The fluidic resistance may be embodied in the form of a flow resistance, for example in the form of a body introduced into the fluid channel and fixed to the fluid channel, or in the form of a contraction of the fluid channel, and/or in the form of a valve, for example a straight-way valve, a corner valve or a slanted seat valve.

The advantages described in connection with the fluid channel system equally apply to the method and will not be repeated below. Features described in connection with the device equally apply to the method.

The inventive method of manufacturing one of the above-described fluid channel systems comprises providing the substrate with the chamber and the at least one fluid channel, filling the chamber with photopolymerizable hydrogel through the at least one fluid channel, selectively exposing said hydrogel, in particular using a shadow mask so that hydrogel located in the chamber is at least partially photopolymerized, and flushing each fluid channel for removing non-polymerized hydrogel.

A further inventive method of manufacturing one of the above-described fluid channel systems comprises providing the substrate with the chamber and the at least one fluid channel, filling the chamber with photodepolymerizable hydrogel through the at least one fluid channel, polymerizing said hydrogel, selectively exposing said hydrogel, in particular using a shadow mask so that hydrogel located in each chamber is photodepolymerized, and flushing each fluid channel for removing non-polymerized hydrogel.

It should be noted here that these two methods each relate to the variants of the fluid channel system with a chamber and at least one fluid channel.

A further inventive method of manufacturing one of the above-described fluid channel systems comprises providing the substrate with the hollow space, filling said hollow space with photopolymerizable hydrogel, selectively exposing said hydrogel, in particular using a shadow mask so that hydrogel located in the hollow space is at least partially photopolymerized, such that said at least one fluid channel and the coherent hydrogel structure are formed, and flushing each fluid channel for removing non-polymerized hydrogel.

A further inventive method of manufacturing one of the above-described fluid channel systems comprises providing the substrate with the hollow space, filling said hollow space with photodepolymerizable hydrogel, polymerizing said hydrogel, selectively exposing said hydrogel, in particular using a shadow mask so that hydrogel located in the hollow space is at least partially photodepolymerized, such that the at least one fluid channel and the coherent hydrogel structure are formed, and flushing each fluid channel for removing non-polymerized hydro gel.

It should be noted here that these two methods each relate to the variants of the fluid channel system with a hollow space in which hydrogel is arranged that is structured such that at least one fluid channel and a coherent hydrogel structure are formed. The selective exposure relates to spatially selective exposure.

Photopolymerization or photodepolymerization is advantageous in that the hydrogel may be polymerized at a selectable point in time within a short time and in a spatially defined manner.

The formation of the at least one fluid channel and the hydrogel structure may be effected in any order.

Flushing may be accomplished by means of a flushing liquid, for example distilled water or a flushing buffer, for example phosphate-buffered saline solution (PBS), or by replacing the non-polymerized hydrogel by a liquid, for example a buffer, in particular PBS, or cell or nutrient medium, in particular Dulbecco's Modified Eagle's Medium (DMEM). The (flushing) liquid may be introduced into each fluid channel through one of the two openings to the outside, in particular under pressure, and the non-polymerized hydrogel may be flushed out of each fluid channel through the second opening to the outside.

The shadow mask may be embodied in the form of a flat plate of a material that is transparent at least to UV radiation. The shadow mask may be embodied as a binary mask which is coated with a non-transparent layer in some areas. The shadow mask may be a shadow mask known from the field of photolithography. For example, it may be a chromium mask, i. e. a mask with a chromium layer on a glass plate (chrome on glass, COG), or a mask with an opaque MoSi layer on a glass plate (opaque MoSi on glass, OMOG). As an alternative, a phase mask may also be used.

Exposure may be effected by means of a light source, for example a UV lamp. In particular, a light source having a wavelength within a range of 315 nm to 600 nm, in particular 340 nm to 400 nm, in particular 365 mm may be used. The power may be between 1 mW/cm² and 100 mW/cm², in particular between 1 mW/cm² and 20 mW/cm², in particular between 4 mW/cm² and 10 mW/cm². Typical durations of exposure are 1 s to 420 s, in particular 1 s to 60 s, in particular 5 s to 30 s, the duration of exposure being lower the higher the power is.

The light may strike on the hydrogel perpendicularly or obliquely. With a perpendicular incidence of light, hydrogel structures such as cuboids or cubes may be generated. With an oblique incidence of light, hydrogel structures with oblique sidewalls may be produced. An optical set-up for parallelizing light or an optical set-up for focusing light may be provided. Parallel light permits a precise reproduction of the structure of the shadow mask onto the hydrogel. The light may also be focused. For example, light that shines in from the bottom in parallel may be focused by means of a converging lens onto the focus that is situated on the surface of the non-polymerized gel. If circular opaque regions are used on the mask, for example cones or truncated cones of polymerized hydrogel are formed.

Exposure may be accomplished perpendicularly or obliquely from the bottom. The shadow mask is arranged over the light source, and optionally over the optical set-up, and the substrate filled with non-polymerized hydrogel is disposed over it. As an alternative, exposure may be effected perpendicularly or obliquely from the top or from the side. The light may be guided through further optical elements, for example deflection mirrors. This may permit to design the optical arrangement to be more compact or in any other advantageous way. It is advantageous to bring the mask as close to the substrate as possible during exposure, in particular into direct contact with the substrate, optionally even with application of pressure. Thus, a good optical reproduction of the structures of the mask on the hydrogel is obtained.

Exposure may be effected such that the hydrogel in the chamber or the hydrogel structure is structured, in particular structured such that channels are formed in the hydrogel, in particular channels that connect the first fluid channel and the second fluid channel or the two partial areas of the single fluid channel. As an alternative or in addition, hollow spaces may be formed in the hydrogel which may in particular be connected to each other.

The mask may be designed such that the hydrogel in each fluid channel is not exposed. For this, a photopolymerizable hydrogel may be used. Thus, each fluid channel is free from hydrogel after flushing. The mask may alternatively be designed such that the hydrogel in each fluid channel is exposed. For this, a photodepolymerizable hydrogel may be used. Thus, each fluid channel is free from hydrogel after flushing.

The level of the hydrogel may be controlled by the amount of the introduced non-polymerized hydrogel, i. e. by means of the filling level.

The formation of the at least one fluid channel may comprise a formation of exactly one, at least two, in particular exactly two fluid channels.

The method may comprise, before the filling, a complete or partial functionalization of the surface of the substrate to bind the hydrogel, in particular chemically, for example covalently, to the surface of the substrate, in particular to the walls of the chamber. The binding may in particular be a specific binding. The binding may prevent the detachment of hydrogels from the substrate.

In particular, binding molecules, for example heterobifunctional PEG-linkers or functionalized silanes, may be applied onto the surface of the substrate which covalently bind at the surface of the substrate. The binding molecules may comprise the same binding groups at the surface which are used in the hydrogel for photopolymerization. In this case, during photopolymerization, a covalent binding to the surface is simultaneously formed. This means, the binding of the hydrogel to the surface may be effected in the same step as photopolymerization. However, it is also possible to carry out photopolymerization and the binding to the surface independently, for example by using chemistry for the binding to the surface which is independent of the cross-linking of the hydrogel. So, the binding of the hydrogel to the surface may be effected in a separate step.

The photopolymerizable or the photodepolymerizable hydrogel may be a first photopolymerizable hydrogel or a first photodepolymerizable hydrogel, wherein the chamber is not completely filled with polymerized hydrogel after the filling, exposure and flushing of the first polymerizable hydrogel or the first photodepolymerizable hydrogel. The method may subsequently comprise filling the chamber with a second hydrogel through the at least one fluid channel, wherein said second hydrogel is photopolymerizable or photodepolymerizable. The method may subsequently comprise a selective exposure of the second hydrogel, in particular using a shadow mask, so that non-polymerized hydrogel located in the chamber is at least partially photopolymerized, or second hydrogel located in each fluid channel is photodepolymerized, in particular completely. The method may subsequently comprise flushing each of the channels for removing non-polymerized second hydrogel.

The method may comprise the repetition of the above-described methods with various hydrogels until the chamber is filled with polymerized hydrogel to at least 90% of capacity, in particular at least 95%, in particular completely.

In the method, the, or at least one of the, photopolymerizable hydrogels or the photodepolymerizable hydrogels may comprise cells when the chamber is being filled. As an alternative, cells may only be introduced after photopolymerization or photodepolymerization of the hydrogel, for example by establishing a contact between the hydrogel and a liquid containing cells. For this, the liquid that contains cells may be introduced into the at least one fluid channel.

The invention also provides a use of one of the above-described fluid channel systems.

The use of one of the above-described fluid channel systems comprises filling the, or a first one of the two fluid channels through the opening to the outside with a liquid containing particles which are designed such that they may penetrate the hydrogel, in particular particles interacting with particles or cells contained in the hydrogel.

The liquid may be, for example, a cell medium, in particular DMEM. The liquid may contain, for example, nutrients, in particular glucose, glutamate, or pyruvate, and/or gases dissolved in the medium, for example oxygen, in particular nutrients or gases which are required for keeping cells alive and/or for promoting cell growth. The supply of the cells may be effected in particular statically, i.e. exclusively via diffusion, or by a continuous flow of the liquid through the fluid channel, through one of the fluid channels or through the fluid channels.

The use of the fluid channel system may comprise filling the second one of the two fluid channels with a second liquid through the opening to the outside. The second liquid may equally comprise the particles contained in the first liquid, in particular at a lower concentration than in the first liquid, and/or other particles or no particles.

The use of the fluid channel system may furthermore comprise the application of pressure to the one or to a first and/or a second one of the two fluid channels. The pressure may be, for example, between 0.1 mbar and 300 mbar, in particular 5 mbar and 70 mbar, in particular between 5 mbar and 60 mbar. The application of pressure creates a so-called interstitial flow through the hydrogel which similarly also occurs in biological tissue.

Depending on the strength of the metabolism and the number or density of cells in the hydrogel, a supply of the cells only by diffusion of the nutrients and gases dissolved in the medium is not sufficient. By the interstitial flow, even larger cell accumulations or even tissue sections of organisms may be kept alive for extended periods.

The use of the fluid channel system may additionally comprise the application of pressure to the second one of the two fluid channels, the pressure being unequal to the pressure in the first one of the two fluid channels. In particular, the pressure in the fluid channel with a higher particle concentration may be higher than the pressure in the fluid channel with a lower particle concentration of the same particles.

The first fluid channel may be filled with a first liquid, and the second fluid channel may be filled with a second liquid. In the first liquid, particles may be contained which are not contained in the second liquid, and in the second liquid, particles may be contained which are not contained in the first liquid, in particular particles having a different effect on cells than the particles in the first liquid. One may control which particles penetrate the hydrogel or in which ratio the various particles penetrate the hydrogel according to the fluid channel to which a higher pressure is applied.

The use of the fluid channel system may comprise the application of pressure to the fluid channel(s) in each case using a pump, in particular an air pump, which is connected to one of the openings to the outside of the respective fluid channel. The use of the fluid channel system may furthermore comprise closing one of the at least two openings to the outside of the respective fluid channel each when pressure is applied.

The use of the fluid channel system may comprise supplying cells in the hydrogel with particles in the liquid, and examining the cells, in particular their growth and/or migration, in particular by means of optical microscopy or fluorescence microscopy. It may be observed, for example, how far cells migrate into a cell-free hydrogel per time unit. From these data, parameters, such as the migration speed and the invasiveness of the cells, may be determined. After particles have been added, one can measure their influence on the corresponding parameters.

A further possible use of the fluid channel system is to fill endothelial cells or epithelial cells as a suspension in a buffer into the at least one fluid channel and to observe them. The cells settle on the hydrogel walls and thereby form a biologically relevant limitation to the hydrogel region. Thereby, the fluid channel simulates blood vessels with similar barrier properties as the biological blood vessel walls.

The fluid channel system may be used for examining tumor cells and for establishing models for cell growth, proliferation and/or cell migration. Moreover, efficacy studies for the treatment of tumors may be carried out.

Further features and advantages will be described below with reference to the exemplary figures.

### BRIEF DESCRIPTION OF THE DRAWINGS

In the figures:

FIG. 1 shows a schematic, not drawn to scale bottom view onto the cover plate of a substrate of a first embodiment;

FIG. 2 shows a schematic, not drawn to scale plan view onto the cover plate of a substrate of the first embodiment;

FIG. 3 shows a schematic, not drawn to scale representation of a section through the fluid channel system of the first embodiment;

FIG. 4 shows a schematic, not drawn to scale diagonal view onto the fluid channel system of the first embodiment;

FIG. 5 shows a schematic, not drawn to scale bottom view onto the cover plate of a substrate of a second embodiment;

FIG. 6 shows a schematic, not drawn to scale bottom view onto the cover plate of a substrate of a third embodiment.

FIGS. 7 to 11 show a schematic, not drawn to scale representation of a manufacturing method for a fluid channel system according to a first embodiment;

FIGS. 12 to 14 show schematic, not drawn to scale representations of further variants of the manufacturing method for a fluid channel system according to the first embodiment;

FIGS. 15 to 17 show a schematic, not drawn to scale representation of a manufacturing method for a fluid channel system according to a second embodiment;

FIG. 18 shows a schematic, not drawn to scale representation of a shadow mask for manufacturing angled fluid channels;

FIG. 19 shows a schematic, not drawn to scale representation of a shadow mask which is inverse to the shadow mask of FIG. 8;

FIG. 20 shows a schematic, not drawn to scale representation of a first embodiment for the use of the device; and

FIG. 21 shows a schematic, not drawn to scale representation of a second embodiment for the use of the device.

### DETAILED DESCRIPTION

Below and in the figures, the same reference numerals are used for the same or corresponding elements, an exception being the increment of the hundreds digit, if not otherwise specified.

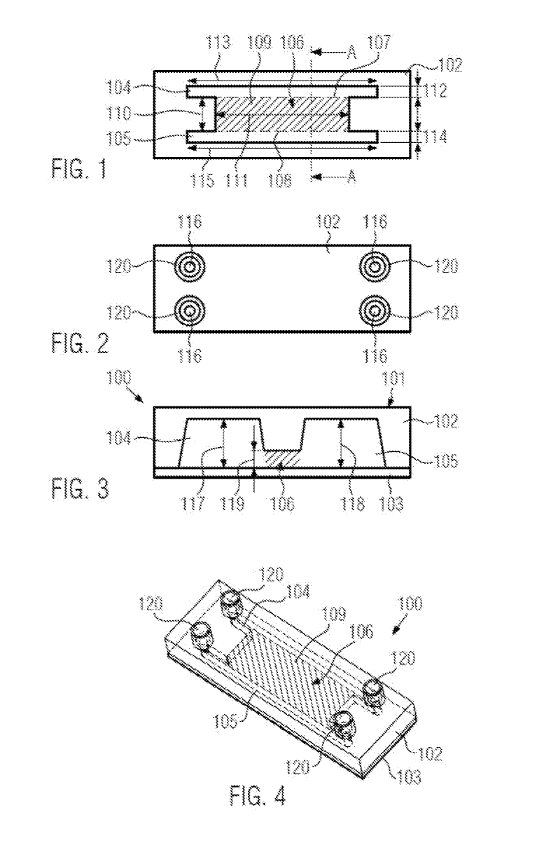

FIGS. 1 to 4 show various elements of the inventive fluid channel system 100 according to a first embodiment. In particular, FIG. 1 shows a part of a plate-like, transparent plastic substrate 101, in this case the cover plate 102 of the plastic substrate, in a view from the bottom, and FIG. 2 shows the cover plate 102 in a plan view. FIG. 3 shows a cross-section of the fluid channel system with the cover plate 102 and the base plate 103 of the substrate fixed thereto. The cover plate of the plastic substrate is embodied in the form of a plastic cuboid in which in turn indentations are formed that are covered by the essentially flat base plate, so that a first fluid channel 104, a second fluid channel 105 and a chamber 106 are formed in the substrate. As can be seen in the bottom view in FIG. 1, the chamber has exactly one opening 107 to the first fluid channel and exactly one opening 108 to the second fluid channel. It is open towards the first fluid channel and the second fluid channel each along its complete length. The chamber, however, may also have several openings towards the first and/or second fluid channel each, it may have, for example, walls interrupted towards the fluid channel.

In a plan view, the chamber has a rectangular contour. It may also have, however, a square, otherwise polygonal, round or oval contour, for example. The length 111 of the chamber is in this example greater than the width 110 of the chamber, so the ratio of the width to the length of the chamber is smaller than 1.

In the chamber, a photopolymerized hydrogel 109 is arranged which extends over the complete width and length of the chamber. However, this does not have to be the case. For example, the hydrogel may not extend to all walls. So, it may have, for example, a shorter length than the length of the chamber. Nevertheless, the chamber must be filled with hydrogel to at least 90% of capacity. In the chamber, several different photopolymerized hydrogels may also be arranged which together fill the chamber to at least 90% of capacity.

It should be noted that, for example due to the manufacturing method, the height of the hydrogel in the chamber may be greater in the region of the walls of the chamber than in regions that are spaced apart from the walls, for example by formation of a meniscus at the walls of the chamber.

The hydrogel in this example prevents a free flow between the two fluid channels or from one partial area of a single fluid channel to the other partial area of the single fluid channel through the chamber. As an alternative, the hydrogel may be embodied such that a liquid filled into one of the two fluid channels or into a partial area of the fluid channel may flow through the chamber into the other fluid channel or into the other partial area of the fluid channel.

The chamber (or the later described hydrogel structure) may each also be referred to as observation region since it typically contains cells which are observed when using the fluid channel system.

The selection of the height, width, length and/or a certain volume of the chamber (or the later described hydrogel structure) moreover permits to imitate certain boundary conditions in cell growth. Depending on the size of the chamber, for example models for small tumors well supplied with nutrients (diameter of the microtumors maximally 1 mm) may be developed, or else models for larger tumors, i. e. tumors that are partially not sufficiently supplied with nutrients (tumor size up to maximally 5 mm) which are not necrotic inside or develop necrotic tissue inside. The values to be selected depend on the nutrient consumption of the observed cells, i. e. on their metabolism.

As is shown in the figure, the ratio of the width 112 of the first fluid channel to the length 113 of the first fluid channel is smaller than 0.2. However, the ratio may also vary, the fluid channel, however, is preferably longer than wide. The same is true for the width 114 and length 115 of the second fluid channel.

The first fluid channel and the second fluid channel each have a rectangular contour in this embodiment in a plan view. Here, however, other shapes are also possible, for example an angled or bent shape.

In FIG. 2, the base plate 102 of the plastic substrate is shown in a plan view. In the base plate, four through holes are formed of which two each form the openings to the outside 116 of the first and the second fluid channel, respectively. Moreover, reservoirs 120 are shown which are arranged to surround the openings as part of the substrate at its outer side and to which, for example, a tube or a pump may be fixed.

FIG. 3 shows a cross-section along line A-A plotted in FIG. 1 through the inventive fluid channel system of the first embodiment. One can see here that the cross-section both of the first and the second fluid channel and the chamber is in each case trapezoidal. The volume of the chamber or the fluid channel each forms truncated pyramids. However, other cross-sections are also possible, for example a rectangular cross-section.

As is shown here, the base plate 103, which is embodied here, for example, in the form of a foil, has a length and width which correspond to the length and width of the cover plate 102 of the plastic substrate. The foil has a thickness of about 0.2 mm.

The height 117 of the first fluid channel and the height 118 of the second fluid channel each are greater than the height 119 of the chamber. The chamber, however, may also have the same height as the fluid channels. One can also see here that the hydrogel 109 occupies the complete height of the chamber. However, this might not be the case, in which case the chamber, however, must be filled with hydrogel to at least 90% of capacity.

FIG. 4 shows a diagonal view of the above-described embodiment.

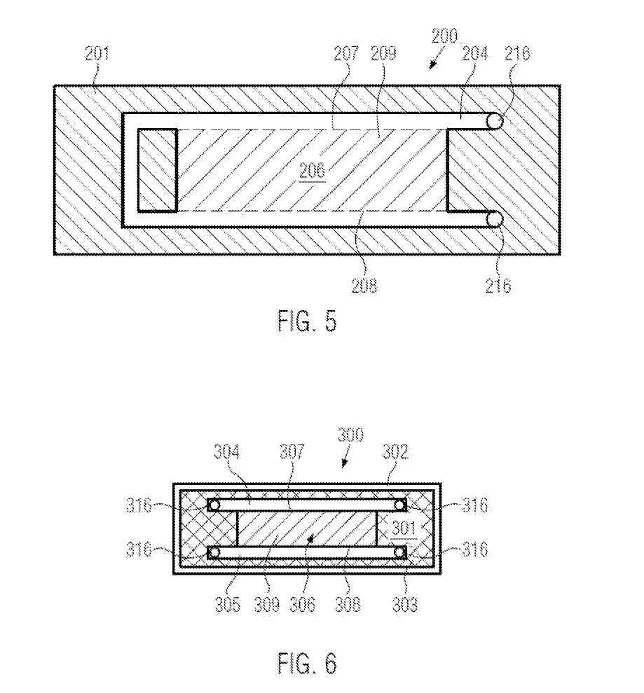

In FIG. 5, the bottom view onto the cover plate of a substrate 201 of a second embodiment 200 is shown. In this case, the fluid channel system comprises exactly one fluid channel 204 towards which the chamber 206 is open exclusively at two opposite partial areas of the fluid channel via the openings 207 and 208 of the chamber. Apart from the design with only one instead of two fluid channels, the fluid channel system is embodied as in the first embodiment. Therefore, here no cross-section, no plan view and no diagonal view are shown anymore. The shape and dimensions of the substrate, the fluid channels and the chamber, however, may also differ from those in the first embodiment. A non-depicted fluidic resistance may be disposed between the two legs of the fluid channel.

In FIG. 6, the bottom view onto the cover plate 302 of a substrate 301 of a third embodiment is shown in which the fluid channel system 300 comprises a hollow space which is limited by the cover plate with side walls and a base plate (not shown here) of glass or plastic. This means, the substrate is a two-piece substrate. A photopolymerized hydrogel 303 is arranged in the hollow space in which a first fluid channel 304 and a second fluid channel 305 are formed. Between the first fluid channel and the second fluid channel, a coherent hydrogel structure 306 is arranged. The hydrogel structure adjoins each with a surface area the first and second fluid channels and is connected to the outside exclusively via the two fluid channels. In the cover plate, four through holes 316 are formed which form the two openings to the outside of the first fluid channel and the two openings to the outside of the second fluid channel.

The fluid channels are embodied corresponding to the first or second embodiment. The hydrogel structure is embodied corresponding to the dimensions of the chamber of the first and second embodiments. The shape and dimensions, however, may also vary.

FIGS. 7 to 11 show the steps of an inventive manufacturing method for a fluid channel system.

FIG. 7 shows a two-piece substrate 401 in a cross-section, for example a plastic or glass substrate, in which two fluid channels 404 and 405 and a chamber 406 are formed which may have, for example, the shapes and dimensions that have been described in the first embodiment of the device. Through the non-depicted openings to the outside of the fluid channels, a liquid, non-polymerized hydrogel is filled into the fluid channels and the chamber until the fluid channels and the chamber are completely filled. If the polymerized hydrogel should not extend up to the top of the chamber, it does not have to be filled in completely.

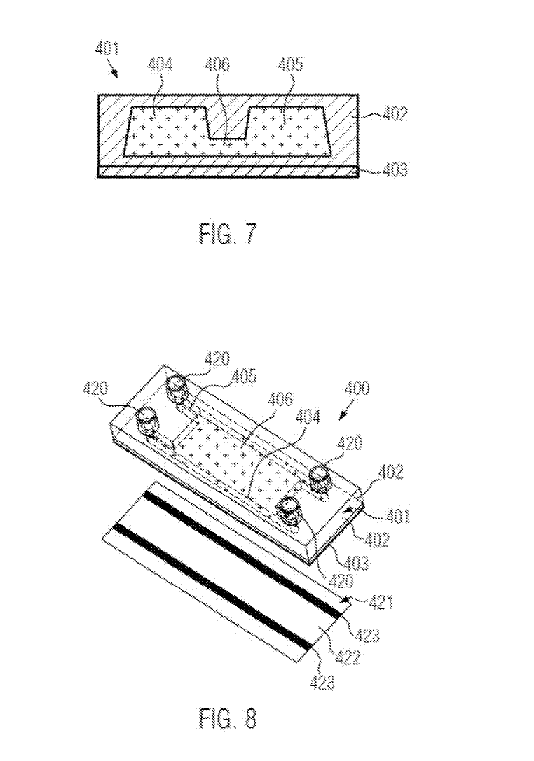

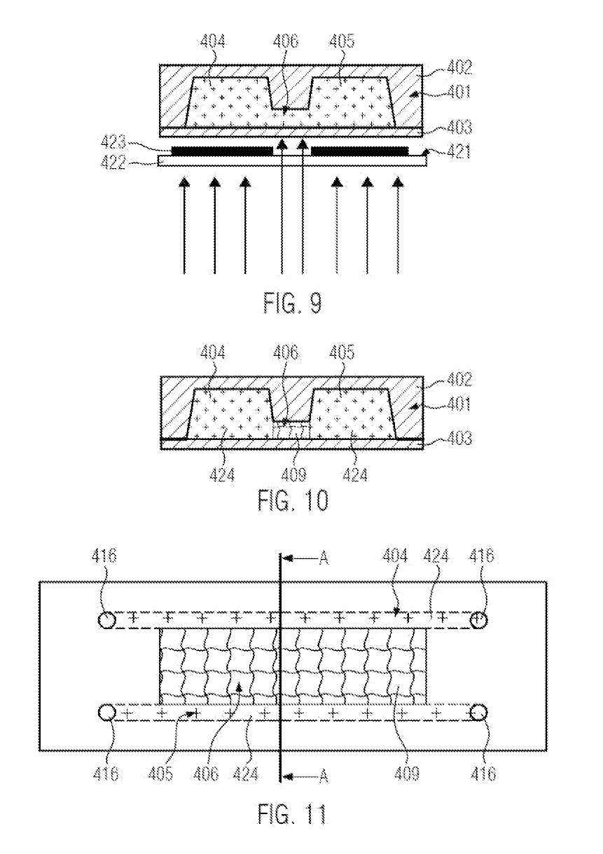

As is shown in FIGS. 8 and 9, the hydrogel is structured by photopolymerization in a next step. FIG. 8 shows, in a diagonal view, how a shadow mask 421 is approached to the substrate from the bottom. In this case, the shadow mask is a chromium mask consisting of a transparent glass plate 422 on which two chromium strips 423 in form of a chromium layer are applied on the glass plate. The chromium strips do not let light pass. However, a chromium mask does not have to be used, any other common type of shadow mask may rather be used. The length and width of the shadow mask here approximately correspond to the length and width of the substrate, but it may also have a different size, for example if this is necessary due to the optical set-up. The chromium strips are shaped and the shadow mask is positioned on the substrate for exposure in such a manner that the chromium strips completely cover both fluid channels but do not cover the chamber.

In FIG. 9, a cross-section along line A-A plotted in FIG. 11 shows how parallel light strikes on the substrate 401 filled with hydrogel from the bottom through the shadow mask 421. As one can see, light only reaches the substrate through the mask where no chromium strips 423 are arranged in the light path (not considering diffraction effects). In this case, only the hydrogel in the chamber is photopolymerized by this. While in the figure, a distance between the mask and the substrate is indicated, in practice, a preferably small distance, in particular a direct contact, is advantageous, as then the structures on the shadow mask may be more precisely imaged on the hydrogel.

FIGS. 10 and 11 each show in a cross-section along line A-A or in a plan view that the hydrogel 409 in the chamber is polymerized while the hydrogel 424 in the fluid channels is not polymerized. In a step which is not shown here, a flushing buffer is introduced into the fluid channels through one opening 416 of each of the two fluid channels and pumped through the fluid channels, so that the non-polymerized hydrogel is displaced by the flushing buffer and removed from the fluid channel through the respective second opening.

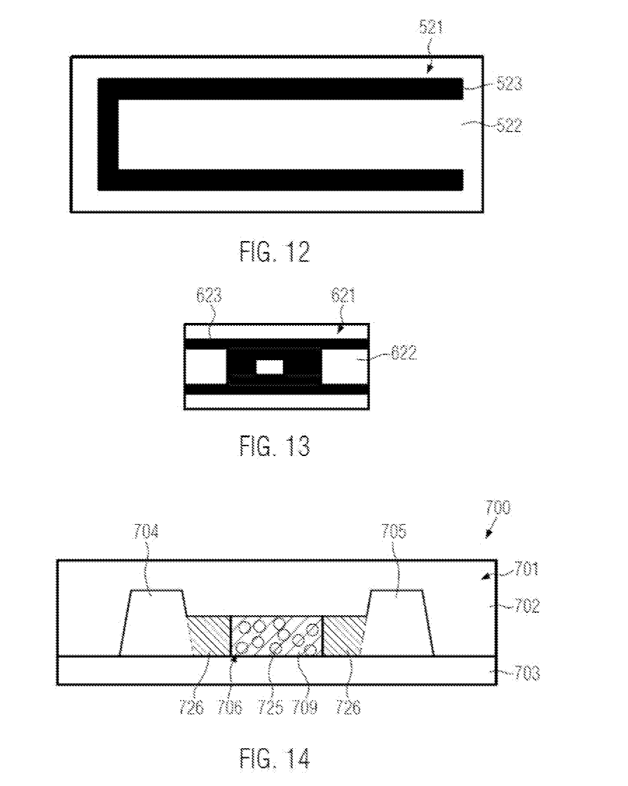

By means of the above-described steps, for example a fluid channel system of the first embodiment may be produced. In a similar manner, a fluid channel system of the second embodiment may be produced, the mask 521 here having, for example, the shape shown in FIG. 12.

In the above-mentioned method, it is also possible for the exposure step to be a first exposure step where a mask 621 different from the mask shown in FIG. 8 is used, where the chromium strips 623 of the mask do not only cover the fluid channels but also a part of the chamber, as is shown, for example, in FIG. 13. So, in this case, the chamber is not completely filled with hydrogel after the exposure step and after flushing. A further step follows the first flushing in which again non-polymerized hydrogel is filled into the fluid channels through the openings to the outside and a second exposure step is carried out with a second mask. In the second mask, chromium strips in turn cover the two fluid channels, however not the chamber or only parts of the chamber. These procedure steps may be repeated until the chamber is filled with hydrogel to at least 90% of capacity.

Such a multi-step method results, for example, in the structure shown in a cross-section in FIG. 14. Here, the hydrogels employed one after the other may have different properties. For example, in the first introduced hydrogel 709, cells 725 may be suspended. In the second hydrogel 726, however, no cells may be suspended. The second hydrogel which adjoins the hydrogel may, for example, differ from the first hydrogel in its migration capability for the cells or in its permeability to liquids. For example, the second hydrogel may be migratable for the cells in the first hydrogel. Adjoining the second hydrogel, a third hydrogel (not shown here) may be arranged which represents a migration barrier for cells.

Such heterogeneous hydrogel structures are particularly suited for examining cell propagation and invasiveness. For example, one can observe with a microscope how the cells penetrate the invasion region. An invasion region is limited, for example, by the above-mentioned third hydrogel. For example, the cells are captured in a predetermined region and available for analysis, in particular “before-after analyses”, so that quantitative statements may be made. For example, a difference is recorded directly after the preparation and after a certain period, so that, for example, the migration speed of the cells may be determined, in particular with or without the addition of migration-promoting or migration-inhibiting substances.

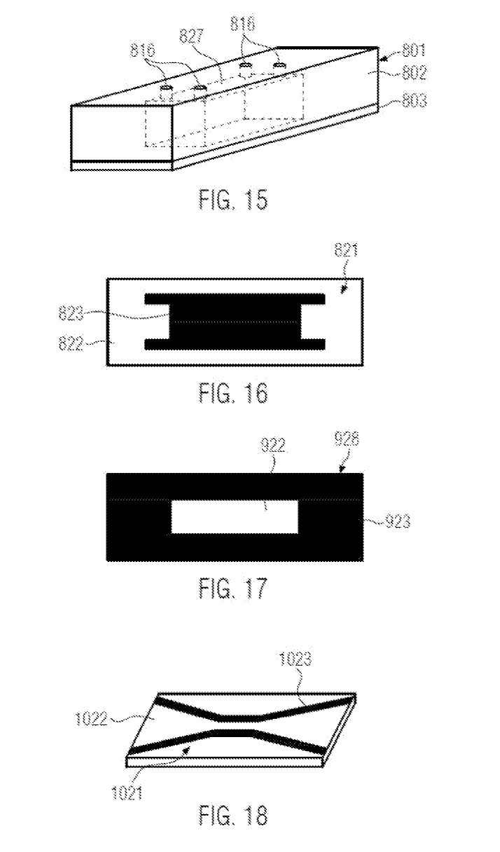

In a further embodiment of a method of manufacturing a fluid channel system which is shown in FIG. 15, a hollow space 827 which does not have any channel structures and no chamber is completely filled with hydrogel. The hollow space is here formed by a two-piece substrate, i. e. by a cover plate 802 comprising an indentation and four through holes as openings 816 to the outside and by a base plate 803. The base plate may be embodied, for example, in the form of a plastic foil.

The liquid hydrogel is filled into the openings 816 to the outside. Subsequently, an exposure through a shadow mask 821 is accomplished, similar as in the previous embodiment. In this example, the shadow mask comprises a chromium layer 823 which covers two parallel strips and a rectangle disposed between the two strips. Such a mask is shown in a plan view in FIG. 16.

If now exposure is effected, for example as in the previous embodiment, the hydrogel is photopolymerized in all regions except for in the regions of the strips and the rectangle. The non-polymerized hydrogel will be removed by flushing. In the process, either openings to the outside may be used which were already present in the substrate, or openings to the outside may be subsequently formed in the substrate. In this manner, two fluid channels and one chamber are formed in the hollow space which may then be processed further as in the first embodiment.

As an alternative, and as described above, the hollow space may be filled with liquid hydrogel, and a mask 928 as shown in FIG. 17 may be used, where a glass plate 922 is completely provided with a chromium layer 923 except for a region centrically arranged with respect to a substrate, for example in the form of a rectangle, so that after exposure and flushing, a cuboid of photopolymerized hydrogel, which forms, for example, the coherent hydrogel structure 306, remains in the center of the hollow space. In a next step, the hollow space may be filled with a second hydrogel, and a shadow mask 421 as in FIG. 8 may be used, so that the hydrogel is photopolymerized in the complete hollow space, except for a region which will later form the fluid channels. Thus, one obtains a fluid channel system as represented in FIG. 6.

FIG. 18 shows an example of a mask 1021, comprising a glass plate 1022 and a chromium layer 1023 by which angled fluid channels may be manufactured with the above-described methods.

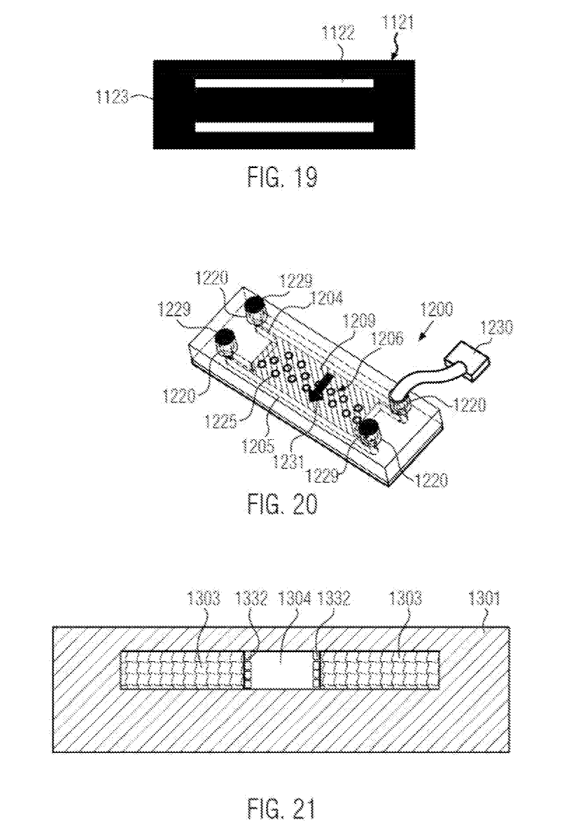

The above-mentioned methods may each also be carried out with a photodepolymerizable hydrogel. For this, the chamber and the at least one fluid channel or the hollow space are filled with the photodepolymerizable hydrogel. This is then polymerized, for example by heating, cooling or changing the pH value. A mask which is, for example, inverse to the above-described masks, is used for the exposure step. Thus, the exposed portion of the polymerized hydrogel is depolymerized again, i. e. the hydrogel in the fluid channel(s), and may be flushed away as described above. As an example, a mask 1121 inverse to the mask shown in FIG. 8 is shown in FIG. 19 which comprises a glass plate 1122 with a chromium layer 1123. During exposure, the hydrogel is depolymerized in the region of the channels. One may also use a mask inverse to the respective masks in FIGS. 12, 13 and 16 to 18 if one works with photodepolymerizable hydrogel in connection with the respective method. These masks are not specifically shown. The fact that the masks are inverse to the masks shown above is optional and only listed as an example for a better understanding. The mask only has to ensure that exclusively selected hydrogel regions from which the hydrogel is to be flushed away subsequently are exposed.

In FIG. 20, a use of the device of FIG. 4 is schematically shown by way of example. The use may also be effected in a similar manner with other devices, in particular the devices shown in FIGS. 5, 6 and 14. Cells 1225 are arranged in the hydrogel 1209 in the chamber 1206. A liquid is filled into the two fluid channels 1104, 1105 through one or both openings 1220 to the outside. In the fluid channel 1204, there is a cell medium for supplying the cells, for example DMEM. The fluid channel 1205 is then closed at both openings to the outside, and the fluid channel 1104 is closed at one opening. For this, plugs 1229 are used, for example. A pump 1230 is connected to the not closed opening of the fluid channel 1204. With this pump, a pressure is applied, so that the pressure in the fluid channel 1204 is higher than the pressure in the fluid channel 1205. Thus, an interstitial flow of the cell medium through the hydrogel is effected (indicated by arrow 1231).

The openings to the outside do not have to be closed in the method. Optionally, a pump may also be connected to the fluid channel 1205. The respective liquid may then also be pumped through the fluid channels, for example.

With a non-depicted microscope or other measuring methods, in particular optical measuring methods, the cells in the hydrogel may be observed, for example in transmission. Thus, cell growth and migration may be examined.

In a further use of a device according to the invention, endothelial cells or epithelial cells 1332 are filled into the fluid channel 1304 as a suspension in a buffer. In this case, the fluid channel is formed in a hydrogel 1303 in the substrate 1301 (for example as the fluid channel system that was described in connection with FIG. 6 and/or a fluid channel system which was manufactured with the method described in connection with FIG. 15). The cells settle on the hydrogel walls, as is shown in the cross-section in FIG. 21, and thereby form a biologically relevant limitation to the hydrogel. Thereby, the fluid channel simulates blood vessels with similar barrier properties as those of biological blood vessel walls. It is also possible for the fluid channel not to be formed in a hydrogel and the endothelial cells or epithelial cells to settle on the walls of the hydrogel in the chamber.

This use may be combined with the above mentioned use. So, the transfer of particles from the blood into tissue may be imitated and examined, for example by microscopy.

It will be understood that features mentioned in the above described embodiments are not restricted to these special combinations and are also possible in any other combinations.

1.-15. (canceled)

16. A fluid channel system for examining cells, comprising:
a chamber filled with at least one photopolymerized hydrogel and/or one polymerized photodepolymerizable hydrogel to at least 90% of capacity,
wherein the chamber is open via at least two openings exclusively towards at least one fluid channel,
wherein the at least one fluid channel and the chamber are each embodied in the form of a hollow space in a substrate, and
wherein each fluid channel has two openings to the outside.

17. The fluid channel system according to claim 16, wherein the chamber is completely filled to capacity with the at least one photopolymerized hydrogel and/or the one polymerized photodepolymerizable hydrogel.

18. The fluid channel system according to claim 16, wherein the at least one fluid channel comprises exactly one fluid channel, and
wherein the chamber is open towards the fluid channel at a first partial area of the fluid channel and a second partial area of the fluid channel.

19. The fluid channel system according to claim 16, wherein the at least one fluid channel comprises a first fluid channel and a second fluid channel, and
wherein the at least two openings of the chamber comprise a first opening to the first fluid channel and a second opening to the second fluid channel.

20. The fluid channel system according to claim 19, wherein the chamber is arranged completely or partially between the first fluid channel and the second fluid channel.

21. The fluid channel system according to claim 16, wherein the at least one hydrogel comprises at least two different hydrogels.

22. The fluid channel system according to claim 16, wherein the at least one of the hydrogels comprises cells.

23. The fluid channel system according to claim 16, wherein the substrate has a one-piece or multi-piece design.

24. A fluid channel system for examining cells, comprising:
a hollow space in a substrate,
wherein in the hollow space, at least one photopolymerized hydrogel and/or one polymerized photodepolymerizable hydrogel is arranged,
wherein the hydrogel is each structured such that at least one fluid channel and a coherent hydrogel structure are formed, and
wherein the hydrogel structure adjoins the at least one fluid channel at two separate surface areas and has a connection to the outside exclusively via the at least one fluid channel.

25. The fluid channel system according to claim 24, wherein the at least one fluid channel comprises exactly one fluid channel, and
wherein one of the two surface areas of the hydrogel structure adjoins a partial area of the fluid channel, and the other one of the two surface areas of the hydrogel structure adjoins a second partial area of the fluid channel.

26. The fluid channel system according to claim 24, wherein the at least one fluid channel comprises a first fluid channel and a second fluid channel, and
wherein the two separate surface areas comprise a first surface area adjoining the first fluid channel and a second surface area adjoining the second fluid channel.

27. The fluid channel system according to claim 24, wherein the hydrogel structure comprises at least two different hydrogels.

28. The fluid channel system according to claim 24, wherein the hydrogel structure comprises cells.

29. The fluid channel system according to claim 24, wherein the substrate has a one-piece or multi-piece design.

30. A method of manufacturing a fluid channel system, comprising:
providing a substrate with a chamber and at least one fluid channel,
filling the chamber with photopolymerizable hydrogel through the at least one fluid channel,
selectively exposing the hydrogel so that hydrogel located in the chamber is at least partially photopolymerized, and
flushing each fluid channel for removing non-polymerized hydrogel.

31. The method according to claim 30, wherein the formation of said at least one fluid channel comprises the formation of exactly two fluid channels.

32. The method according to claim 30, comprising, before the filling, a complete or partial functionalization of the surface of the substrate to bind the hydrogel to the surface of the substrate.

33. The method according to claim 30, wherein the photopolymerizable hydrogel is a first photopolymerizable hydrogel, wherein the chamber is, after the filling, exposing and flushing the first photopolymerizable hydrogel, not completely filled with hydrogel, comprising:
filling the chamber with a second hydrogel through the at least one fluid channel, wherein the second hydrogel is photopolymerizable,
selectively exposing said second hydrogel so that second hydrogel located in the chamber is at least partially photopolymerized, and
flushing each fluid channel for removing non-polymerized second hydrogel.

34. The method according to claim 30, wherein the at least one of the photopolymerizable hydrogels comprises cells during filling.

35. The method according to claim 30, wherein the hydrogel is selectively exposed using a shadow mask.
